(19) United States
(12) Patent Application Publication
(10) Pub. No.: US 2009/0275597 A1
(43) Pub. Date: Nov. 5, 2009

(54) METHODS OF TREATING CNS DISORDERS

(75) Inventors: Kelly PAPADAKIS, Shamong, NJ (US); Nika ADHAM, Englewood Cliff, NJ (US); Gary SAMORISKI, Hillsborough, NJ (US)

Correspondence Address: Forest Laboratories, Inc.;Attn: Charles S. Ryan, 909 3rd Avenue, New York, NY, 10022, US

(73) Assignee: FOREST LABORATORIES HOLDINGS LIMITED, Hamilton (BE)

(21) Appl. No.: 12/433,928

(22) Filed: May 1, 2009

Related U.S. Application Data
(60) Provisional application No. 61/049,905, filed on May 2, 2008.

(51) Int. Cl. A61K 31/4965; A61K 31/13

(52) U.S. Cl. 514/255.03; 514/579; 514/662

(57) ABSTRACT

The present invention relates to methods of treating various CNS disorders, e.g., mania, bipolar disorder and schizophrenia, by administering NMDA receptor antagonists, alone or in combination with dopamine receptor antagonists.

### FIELD OF THE INVENTION

The present invention relates to methods of treating various disorders, e.g., mania, bipolar disorder and schizophrenia, by administering NMDA receptor antagonists, alone or in combination with dopamine receptor antagonists.

### BACKGROUND OF THE INVENTION

Bipolar disorder in the United States affects 5.7 million adults or about 2.6% of the population 18 years of age and older in any given year and has considerable economic impact on our society. In a 1991 study conducted by the U.S. National Institute of Mental Health, an estimated annual cost of $45 billion was attributed to bipolar disorder in the United States alone (Wyatt R J, Henter I., Soc. Psychiatry Psychiatr. Epidemiol., 30(5), 213-9, 1995). In the year 2000, this disorder ranked as the fifth leading cause of disability in adults between the ages of 15 and 44 (World Health Organization, World Health Report 2001, Mental Health: New Understanding, New Hope).

Bipolar disorder is a complex, chronic illness causing dramatic mood swings and unusual shifts in energy and behavior, ultimately resulting in functional impairments. It manifests itself as alterations in mood and energy from euphoria and excitability to depression and psychomotor retardation (Goodwin F K, Jamison K R., Manic-Depressive Illness. New York: Oxford University Press, 642-647, 1990), and is associated with significant morbidity and mortality. Suicide rates within this population are among the highest of all psychiatric illnesses (Müller-Oerlinghausen et al., Lancet, 359 (9302), 241-7, 2002). Bipolar disorder is treated in phases, with each phase presenting its own set of challenges to the treating physician. Bipolar mania accounts for one in seven psychiatric emergencies. Acute manic and mixed episodes are frequently associated with severe behavioral, physical, functional, and cognitive disturbances, all of which can have important personal and social consequences. For the most part, bipolar mania constitutes a medical emergency requiring a hospital admission to ensure the immediate safety of the patient or others and rapid symptomatic relief (Keck, British Medical Journal, 327 (7422), 1002-3, 2003).

A variety of pharmacological agents are currently available for the management of acute mania, including mood stabilizers, anticonvulsants, and antipsychotics, all of which can be used as monotherapy or in combination regimens. In recent years, the atypical antipsychotics (e.g., olanzapine, risperidone, quetiapine, ziprasidone, aripiprazole) have been approved for mania in bipolar disorder. For example, extended release version of quetiapine (Seroquel XR®) was the first medication approved by the FDA for the once-daily acute treatment of both depressive and manic episodes associated with bipolar disorder. First- and second-generation antipsychotics are used in the acute setting in combination with mood stabilizers to achieve a more rapid control of symptoms in severely agitated patients whose treatment also necessitates hospitalization.

Compared to conventional agents, the side effect profile of atypical antipsychotics may be more favorable. However, atypical antipsychotics have been associated with an increased risk of metabolic side effects, including body weight gain, dyslipidemia, glucose intolerance, and type II diabetes. Because of this increased risk, the U.S. Food and Drug Administration (FDA) requires a warning label for diabetes on all atypical antipsychotics. Other side effects commonly associated with currently available treatment options for acute mania in bipolar patients include tremors, psychomotor slowing, cognitive impairment, exacerbation of agitation, nephrotoxicity, altered thyroid function, cataract development, QT effects, sudden cardiac death and sexual dysfunction.

Therefore, despite substantial advances in the pharmacological treatment of bipolar disorder, treatment needs are still not met by currently available therapies and only a low percentage of patients persistently benefit from treatment (Sachs, J. Clin. Psychopharmacol., 23 (3 Suppl 1), S2-8, 2003). A significant percentage of patients do not fully respond to these treatment options and continue to experience subthreshold symptoms and even relapse. This is attributed partly to the lack of efficacy of currently available medications, the production of intolerable side effects, and the increasing therapeutic costs (especially with use of combination regimens). These drawbacks limit their applicability and result or contribute to patient noncompliance. The optimum acute and long-term treatment strategies for acute bipolar mania are not yet established. More effective therapies with improved side effect profiles are still needed to enhance acute, as well as long-term, outcomes in these patients without the possibility of inducing depression or rapid cycling.

Schizophrenia is a lifelong disabling psychiatric disorder with a reported worldwide prevalence of about 1.5%, and an annual incidence of 5 per 10,000 individuals. The disorder usually manifests during adolescence or in young adulthood and the cardinal symptoms fall into three domains: positive symptoms, such as delusions and hallucinations, negative symptoms, such as lack of drive and social withdrawal, and cognitive symptoms, such as problems with attention and memory. These lead to social and occupational dysfunction, which inevitably have a profound effect on the family and the place of the affected individual in wider society. In addition to psychiatric symptoms, patients with schizophrenia are at greater risk for medical comorbidities than the general population.

Current guidelines recommend atypical antipsychotics, including risperidone, olanzapine, quetiapine, ziprasidone, paliperidone and aripiprazole, as first-line treatment for schizophrenia. These drugs can be uniformly characterized by their dual mode of action: in addition to antagonism of the dopamine D₂ receptor, they are also potent inhibitors at the serotonin 5-HT_{2A} receptor.

Although an improvement over the classical neuroleptics, atypical antipsychotics still have shortcomings in the effective management of the disease. In particular, these drugs are associated with a high incidence of side effects (e.g., extrapyramidal symptoms [EPSs] at high dose, sedation, cardiovascular effects such as QTc prolongation, hematologic alterations, effects on sexual function, weight gain, metabolic abnormalities). Furthermore, treatment resistance remains high with 10-30% of patients having little or no response to currently available antipsychotic medications, and up to an additional 30% of patients having only partial treatment response (see, e.g., Lehman et al., Am. J. Psychiatry, 161 (2 Suppl), 1-56, 2004). This has led to the common clinical practice of experimental use of high doses of atypicals, antipsychotic polypharmacy, and augmentation with other psychotropic drugs (see, e.g., Zink et al., Eur. Psychiatry, 19:56-58, 2004; Stahl and Grady, Curr. Med. Chem., 11, 313-27, 2004).

Of the American Psychiatric Association guidelines for the treatment of schizophrenia, 60-70% of patients relapse within 1 year without maintenance treatment and almost 90% relapse within 2 years (see, e.g., Lehman et al., Am. J. Psychiatry, 161 (2 Suppl), 1-56, 2004).

NMDA receptor antagonists potentially have a wide range of therapeutic applications in several central nervous system (CNS) disorders such as Parkinson's disease, Alzheimer's disease (AD), Huntington's disease, amyotrophic lateral sclerosis (ALS), acute neurodegeneration associated with stroke and trauma, epilepsy, drug dependence, depression, anxiety, and chronic pain (Parsons et al., Drug News Perspect., 11:523-533, 1998; Jentsch and Roth, Neuropsychopharmacology, 20: 201-205, 1999; Doble, Therapie, 50: 319-337, 1995).

Memantine (1-amino-3,5-dimethyl adamantane) and neramexane (1-amino-1,3,3,5,5-pentamethylcyclohexane) are analogs of 1-aminocyclo-hexane that function as NMDA receptor antagonists and prevent the pathological activation of NMDA receptors but allow their physiological activity. Memantine and neramexane, as well as other 1-aminoalkyl-cyclohexanes, are systemically-active noncompetitive NMDA receptor antagonists having moderate affinity for the receptor. They exhibit strong voltage dependent characteristics and fast blocking/unblocking kinetics. Memantine hydrochloride is currently available in the U.S. and in over 42 countries worldwide. It is approved for the treatment of moderate to severe Alzheimer's disease in the United States at a dose of up to 20 mg/day (5-10 mg BID).

U.S. Patent Publication No. 2006/0229297 discloses (thio)-carbamoyl-cyclohexane derivatives that are D₃ and D₂ dopamine receptor subtype preferring ligands, having the formula (I):

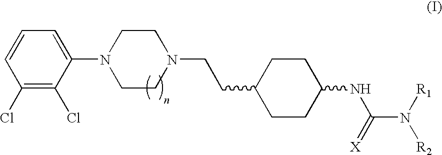

wherein R₁, R₂, X, and n are as defined therein.

One particular compound disclosed therein is trans-1-{4-[2-[4-(2,3-dichlorophenyl)-piperazin-1-yl]-ethyl]-cyclohexyl}-3,3-dimethyl-urea, which is also known as trans-4-{2-[4-(2,3-dichlorophenyl)-piperazin-1-yl]-ethyl}-N,N-dimethylcarbamoyl-cyclohexylamine, the structural formula for which is shown below:

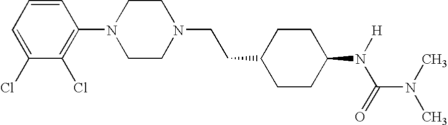

The dopamine D₂ receptors are widely distributed in the brain and are known to be involved in numerous physiological functions and pathological states. D₂ antagonists are widely used drugs as antipsychotics, for example. However, it is also well known that massive antagonism of the D₂ receptors leads to unwanted side-effects such as extrapyramidal motor symptoms, psychomotor sedation or cognitive disturbances. These side effects seriously restrict the therapeutic utilization of D₂ antagonist compounds. (Wong A. H. C. et al.: Neurosci. Biobehav. Rev. 2003, 27, 269.).

The (thio)-carbamoyl-cyclohexane derivatives of formula (I) are orally active and very potent dopamine D₃/D₂ receptor antagonists, which bind with significantly higher potency to D₃ than D₂ receptors. The D₃ receptor antagonism is about one order of magnitude greater than the D₂ receptor antagonism, which is believed to counteract some of the extrapyramidal side effects produced by D₂ receptor antagonists. In addition to the increased relative affinity for dopamine D₃ to D₂, e.g. trans-1-{4-[2-[4-(2,3-dichlorophenyl)-piperazin-1-yl]-ethyl]-cyclohexyl}-3,3-dimethyl-urea hydrochloride has a low potency at other receptor sites such as the 5-HT_{2C}, histamine H₁, and adrenergic receptor sites, which suggest a lower potential for side effects such as EPSs and body weight gain.

The particular combination of the two receptor-actions described above for the derivatives of formula (I) may allow the simultaneous manifestation of the beneficial actions of both the D₃ antagonism (e.g. cognitive enhancer effect, inhibition of extrapyramidal motor symptoms, inhibitory action on drug abuse) and the D₂ antagonism (e.g. antipsychotic effect). Furthermore, the same combination may result in reducing the incidence/severity of disadvantageous features of D₂ antagonism (e.g. extrapyramidal symptoms, psychomotor sedation, cognitive disturbances).

There is an existing and continuing need for new methods of treating disorders such as mania, bipolar disorder and schizophrenia, whereby therapeutic efficacy is maximized and unwanted adverse side effects are decreased.

### SUMMARY OF THE INVENTION

The present invention relates to methods of treating various disorders comprising administering NMDA receptor antagonists, alone or in combination with dopamine receptor antagonists.

In one aspect, the invention relates to methods of treating mania, bipolar disorder or schizophrenia comprising administering an NMDA antagonist. In another aspect, the invention relates to methods of treating mania, bipolar disorder or schizophrenia comprising administering an NMDA antagonist in combination with a dopamine receptor antagonist. According to a further aspect, the invention relates to pharmaceutical compositions comprising an NMDA antagonist and a dopamine receptor antagonist.

### DETAILED DESCRIPTION OF THE INVENTION

In one aspect, the present invention relates to methods of treating a disorder selected from mania, bipolar disorder or schizophrenia, comprising administering to a patient in need thereof a therapeutically effective amount of an NMDA receptor antagonist.

Suitable NMDA receptor antagonists that may be used include, but are not limited to, 1-aminocyclohexane derivatives. The term “1-aminocyclohexane derivative” is used herein to describe a compound which is derived from 1-aminocyclohexane (or an available derivative thereof, such as neramexane or memantine) in the process used to create a similar but slightly different drug.

The 1-aminocyclohexane derivatives as NMDA antagonists for use in the present invention can be represented by the general formula (II):

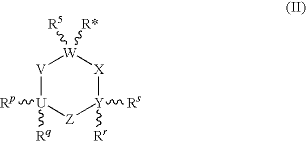

wherein:

R* is -(A)ₙ-(CR¹R²)ₘ—NR³R⁴,

n+m=0, 1, or 2,

A is selected from the group consisting of linear or branched lower alkyl (C₁-C₆), linear or branched lower alkenyl (C₂-C₆), and linear or branched lower alkynyl (C₂-C₆),

R¹ and R² are independently selected from the group consisting of hydrogen, linear or branched lower alkyl (C₁-C₆), linear or branched lower alkenyl (C₂-C₆), linear or branched lower alkynyl (C₂-C₆) aryl, substituted aryl and arylalkyl,

R³ and R⁴ are independently selected from the group consisting of hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene; or independently R³ or R⁴ may join with R^{p}, R^{q}, R^{r}, or R^{s} to form an alkylene chain —CH(R⁶)—(CH₂)ₜ—, wherein t=0 or 1 and the left side of the alkylene chain is attached to U or Y and the right side of the alkylene chain is attached to N and R⁶ is selected from the group consisting of hydrogen, linear or branched lower alkyl (C₁-C₆), linear or branched lower alkenyl (C₂-C₆), linear or branched lower alkynyl (C₂-C₆), aryl, substituted aryl and arylalkyl; or independently R³ or R⁴ may join with R⁵ to form an alkylene chain represented by the formula —CH₂—CH₂—CH₂—(CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N;

R⁵ is independently selected from the group consisting of hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,

R^{p}, R^{q}, R^{r}, and R^{s}, are independently selected from the group consisting of hydrogen, linear or branched lower alkyl (C₁-C₆), linear or branched lower alkenyl (C₂-C₆), linear or branched lower alkynyl (C₂-C₆), cycloalkyl (C₃-C₆) and aryl, substituted aryl and arylaklyl or R^{p}, R^{q}, R^{r}, and R^{s} independently may form a double bond with U or with Y or to which it is attached, or R^{p}, R^{q}, R^{r}, and R^{s} may combine together to represent a lower alkylene —(CH₂)ₙ— or a lower alkenylene bridge wherein x is 2-5, inclusive, which alkylene bridge may, in turn, combine with R⁵ to form an additional lower alkylene —(CH₂)_{y}— or a lower alkenylene bridge, wherein y is 1-3, inclusive,

the symbols U, V, W, X, Y, Z represent carbon atoms,

and include optical isomers, diastereomers, polymorphs, enantiomers, hydrates, pharmaceutically acceptable salts, and mixtures of compounds within formula (I).

The ring defined by U-V-W-X-Y-Z is preferably selected from the group consisting of cyclohexane, cyclohex-2-ene, cyclohex-3-ene, cyclohex-1,4-diene, cyclohex-1,5-diene, cyclohex-2,4-diene, and cyclohex-2,5-diene.

Non-limiting examples of 1-aminocyclohexane derivatives used of the invention include:
1-amino-1,3,5-trimethylcyclohexane,
1-amino-1 (trans),3 (trans),5-trimethylcyclohexane,
1-amino-1 (cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-13,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1-amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3 (cis)-propylcyclohexane,
1-amino-1,3-dimethyl-3-ethylcyclohexane,
1-amino-1,3,3-trimethylcyclohexane,
cis-3-ethyl-1 (trans)-3 (trans)-5-trimethylcyclohexamine,
1-amino-1,3 (trans)-dimethylcyclohexane,
1,3,3-trimethyl-5,5-dipropylcyclohexylamine,
1-amino-1-methyl-3 (trans)-propylcyclohexane,
1-methyl-3 (cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5,5-pentamethylcyclohexane,
1-amino-1-methyl cyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl-1)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,sS)trans-5-ethyl-1,3,3-trimethylcyclohexane,
1-amino-(1S,SS)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3 (cis)-ethyl-cyclohexane,
1-amino-1-methyl-3 (cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,SS)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,SS)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,

their optical isomers, diastereomers, enantiomers, hydrates, their pharmaceutically acceptable salts, and mixtures thereof.

Certain 1-aminocyclohexane derivatives of general formula (II) including the case where three axial alkyl substituents, e.g., R^{p}, R^{r} and R⁵ all together form a bridgehead to yield compounds (so called 1-aminoadamantanes) are illustrated by the formulae IIb-IId below:

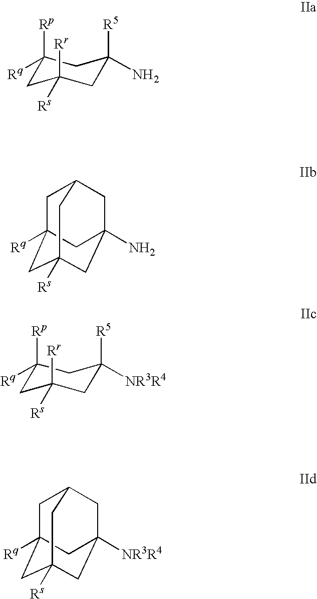

The 1-amino adamantane derivatives of formulae IIb and IId, including memantine, are generally prepared by alkylation of halogenated adamantanes, preferably bromo- or chloroadamantanes. The di- or tri-substituted adamantanes are obtained by additional halogenation and alkylation procedures. The amino group is introduced either by oxidation with chromiumtrioxide and bromination with HBr or bromination with bromine and reaction with formamide followed by hydrolysis. The amino function can be alkylated of generally-accepted methods. Methylation can, for example, be effected by reaction with chloromethyl formate and subsequent reduction. The ethyl group can be introduced by reduction of the respective acetamide. For more details on synthesis, see, e.g., U.S. Pat. Nos. 5,061,703 and 6,034,134. Additional synthetic techniques for the foregoing compounds can be found in U.S. Pat. Nos. 6,828,462 and 7,022,729.

Certain 1-aminocyclohexane derivatives of formula (II) wherein n+m=0, U, V, W, X, Y and Z form a cyclohexane ring, and one or both of R³ and R⁴ are independently joined to said cyclohexane ring via alkylene bridges formed through R^{p}, R^{q}, R^{r}, R^{s} or R⁵ are represented by the following formulae IIIa-IIIc:

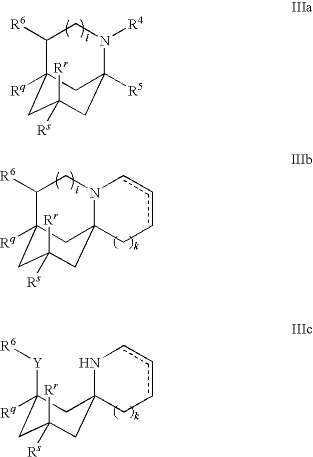

where R^{q}, R^{r}, R^{s}, R^{r} and R⁵ are as defined above for formula (I), R⁶ is hydrogen, linear or branched lower alkyl (C₁-C₆), linear or branched lower alkenyl (C₂-C₆), linear or branched lower alkynyl (C₂-C₆), aryl, substituted aryl or arylalkyl Y is saturated or may combine with R⁶ to form a carbon-hydrogen bond with the ring carbon to which it is attached, 1=0 or 1 and k=0, 1 or 2 and represents a single or double bond.

Additional non-limiting examples of 1-aminocyclohexane derivatives used of the invention include:
1-amino-3-phenyl adamantane,
1-amino-methyl adamantane,
1-amino-3,5-dimethyl adamantane (memantine),
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-N-pyrrolidino and 1-N-piperidine derivatives,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,

their optical isomers, diastereomers, enantiomers, hydrates, N-methyl, N,N-dimethyl, N-ethyl, N-propyl derivatives, their pharmaceutically acceptable salts, and mixtures thereof.

In certain embodiments, the NMDA receptor antagonist is memantine, or a pharmaceutically acceptable salt thereof (e.g., memantine hydrochloride), or neramexane, or a pharmaceutically acceptable salt thereof (e.g., neramexane hydrochloride, neramexane mesylate).

Memantine (1-amino-3,5-dimethyl adamantane) is the subject matter of U.S. Pat. Nos. 4,122,193 and 4,273,774. Neramexane (1-amino-1,3,3,5,5-pentamethylcyclohexane) is disclosed, e.g., in U.S. Ser. No. 09/597,102 and U.S. Pat. No. 6,034,134.

In one embodiment, the present invention relates to methods of treating mania comprising administering to a patient in need thereof a therapeutically effective amount of an NMDA receptor antagonist (e.g., memantine). In other embodiments, the present invention relates to methods of treating acute mania comprising administering to a patient in need thereof a therapeutically effective amount of an NMDA receptor antagonist (e.g., memantine). In other embodiments, the present invention relates to methods of treating acute mania associated with bipolar disorder (e.g., bipolar disorder I or II) comprising administering to a patient in need thereof a therapeutically effective amount of an NMDA receptor antagonist (e.g., memantine)

In other embodiments, the present invention relates to methods of treating bipolar I disorder comprising administering to a patient in need thereof a therapeutically effective amount of an NMDA receptor antagonist (e.g., memantine). In other embodiments, the present invention relates to methods of treating bipolar II disorder comprising administering to a patient in need thereof a therapeutically effective amount of an NMDA receptor antagonist (e.g., memantine). In other embodiments, the present invention relates to methods of treating cyclothymia comprising administering to a patient in need thereof a therapeutically effective amount of an NMDA receptor antagonist (e.g., memantine).

In a further aspect, the present invention relates to methods of treating a disorder selected from mania, bipolar disorder and schizophrenia, comprising administering to a patient in need thereof an NMDA receptor antagonist in combination with a compound of formula (I):

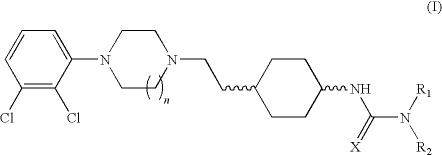

wherein
R₁ and R₂ are each, independently, hydrogen, alkyl, alkenyl, aryl, cycloalkyl or aroyl, or R₁ and R₂ form a heterocyclic ring with the adjacent nitrogen atom;
X is O or S;
n is 1 or 2;
   and/or geometric isomers and/or stereoisomers and/or diastereomers and/or salts and/or hydrates and/or solvates and/or polymorphs thereof.

In certain embodiments, when R₁ and/or R₂ represent alkyl, the alkyl moiety is a substituted or unsubstituted saturated hydrocarbon radical which may be straight-chain or branched-chain and contains about 1 to about 6 carbon atoms (e.g., 1 to 4 carbon atoms), and is optionally substituted with one or more C₁₋₆ alkoxycarbonyl, aryl (e.g., phenyl) or (C₁₋₆ alkoxycarbonyl)-C₁₋₆ alkyl groups, or combinations thereof.

In additional embodiments, R₁ and R₂ form a heterocyclic ring with the adjacent nitrogen atom, which may be a saturated or unsaturated, optionally substituted, monocyclic or bicyclic ring, which may contain further heteroatoms selected from O, N, or S. For example, the heterocyclic ring can be pyrrolidine, piperazine, piperidine or morpholine.

In additional embodiments, when R₁ and/or R₂ represent alkenyl, the alkenyl moiety may have 2 to 7 carbon atoms and 1 to 3 double bonds.

In additional embodiments, when R₁ and/or R₂ represent aryl, the aryl moiety may be selected from an optionally substituted mono-, bi- or tricyclic aryl, such as, but not limited to, phenyl, naphthyl, fluorononyl, or anthraquinonyl group (e.g., phenyl or naphthyl). The aryl moiety may be substituted with one or more C₁₋₆ alkoxy, trifluoro-C₁₋₆ alkoxy, C₁₋₆ alkoxycarbonyl, C₁₋₆ alkanoyl, aryl, C₁₋₆ alkylthio, halogen, cyano groups or combinations thereof.

In additional embodiments, when R₁ and/or R₂ represent cycloalkyl, the cycloalkyl moiety may be selected from an optionally substituted mono-, bi- or tricyclic cycloalkyl group, such as cyclohexyl or adamantyl.

In additional embodiments, when R₁ and/or R₂ represent aroyl the aryl moiety therein is as defined above, e.g., phenyl.

In one embodiment, the compound of formula (I) is trans-1-{4-[2-[4-(2,3-dichlorophenyl)-piperazin-1-yl]-ethyl]-cyclohexyl}-3,3-dimethyl-urea, or a pharmaceutically acceptable salt thereof. For example, the compound of formula (I) is trans-1-{4-[2-[4-(2,3-dichlorophenyl)-piperazin-1-yl]-ethyl]-cyclohexyl}-3,3-dimethyl-urea hydrochloride (i.e., cariprazine hydrochloride).

In other embodiments, the NMDA receptor antagonist is memantine, or a pharmaceutically acceptable salt thereof (e.g., memantine hydrochloride), or neramexane, or a pharmaceutically acceptable salt thereof (e.g., neramexane hydrochloride, neramexane mesylate).

In certain embodiments, the present invention relates to methods of treating a disorder selected from mania, bipolar disorder and schizophrenia comprising administering to a patient in need thereof an NMDA receptor antagonist selected from memantine, neramexane, and pharmaceutically acceptable salts thereof, in combination with trans-1-{4-[2-[4-(2,3-dichlorophenyl)-piperazin-1-yl]-ethyl]-cyclohexyl}-3,3-dimethyl-urea, or a pharmaceutically acceptable salt thereof.

In an exemplary embodiment, the present invention relates to methods of treating a disorder selected from mania, bipolar disorder and schizophrenia comprising administering to a patient in need thereof memantine hydrochloride and trans-1-{4-[2-[4-(2,3-dichlorophenyl)-piperazin-1-yl]-ethyl]-cyclohexyl}-3,3-dimethyl-urea hydrochloride (cariprazine hydrochloride).

In another exemplary embodiment, the present invention relates to methods of treating a disorder selected from mania, bipolar disorder and schizophrenia comprising administering to a patient in need thereof neramexane hydrochloride and trans-1-{4-[2-[4-(2,3-dichlorophenyl)-piperazin-1-yl]-ethyl]-cyclohexyl}-3,3-dimethyl-urea hydrochloride.

In another exemplary embodiment, the present invention relates to methods of treating a disorder selected from mania, bipolar disorder and schizophrenia comprising administering to a patient in need thereof neramexane mesylate and trans-1-{4-[2-[4-(2,3-dichlorophenyl)-piperazin-1-yl]-ethyl]-cyclohexyl}-3,3-dimethyl-urea hydrochloride.

In one embodiment, the combination of an NMDA receptor antagonist (e.g., memantine) and a dopamine receptor antagonist (e.g., cariprazine) may be used to treat cognitive symptoms of schizophrenia. In another embodiment, the combination of an NMDA receptor antagonist and a dopamine receptor antagonist may be used to treat positive symptoms of schizophrenia. In a further embodiment, the combination of an NMDA receptor antagonist and a dopamine receptor antagonist may be used to treat negative symptoms of schizophrenia.

In other embodiments, the combination of an NMDA receptor antagonist (e.g., memantine) and a dopamine receptor antagonist (e.g., cariprazine) may be used to treat affective symptoms of schizophrenia, residual symptoms of schizophrenia, schizoaffective disorder or schizophreniform disorder.

In other embodiments, the present invention relates to methods of treating mania comprising administering to a patient in need thereof a therapeutically effective amount of an NMDA receptor antagonist (e.g., memantine) and a dopamine receptor antagonist (e.g., cariprazine). In other embodiments, the present invention relates to methods of treating acute mania comprising administering to a patient in need thereof a therapeutically effective amount of an NMDA receptor antagonist (e.g., memantine) and a dopamine receptor antagonist (e.g., cariprazine). In other embodiments, the present invention relates to methods of treating acute mania associated with bipolar disorder (e.g., bipolar disorder I or II) comprising administering to a patient in need thereof a therapeutically effective amount of an NMDA receptor antagonist (e.g., memantine) and a dopamine receptor antagonist (e.g., cariprazine).

In other embodiments, the present invention relates to methods of treating bipolar I disorder comprising administering to a patient in need thereof a therapeutically effective amount of an NMDA receptor antagonist (e.g., memantine) and a dopamine receptor antagonist (e.g., cariprazine). In other embodiments, the present invention relates to methods of treating bipolar II disorder comprising administering to a patient in need thereof a therapeutically effective amount of an NMDA receptor antagonist (e.g., memantine) and a dopamine receptor antagonist (e.g., cariprazine). In other embodiments, the present invention relates to methods of treating cyclothymia comprising administering to a patient in need thereof a therapeutically effective amount of an NMDA receptor antagonist (e.g., memantine) and a dopamine receptor antagonist (e.g., cariprazine).

In further embodiments, the secondary social and occupational dysfunctions of schizophrenia are treated. In additional embodiments, the cognitive defects associated with schizophrenia are treated.

Additional disorders which may be treated using a combination of an NMDA receptor antagonist and a dopamine receptor antagonist include, but are not limited to, mild-to-moderate cognitive deficits, dementia, psychotic states associated with dementia, psychotic depression, and paranoid and delusional disorders.

In yet a further aspect, the present invention relates to pharmaceutical compositions containing NMDA receptor antagonists and dopamine receptor ligands (e.g., dopamine D₂/D₃ receptor ligands).

In certain embodiments, the present invention relates to pharmaceutical compositions comprising an NMDA receptor antagonist and a compound of formula (I):

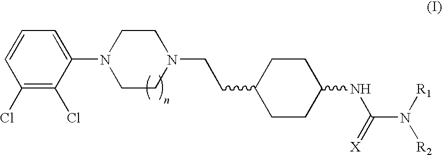

wherein
R₁ and R₂ are each, independently, hydrogen, alkyl, alkenyl, aryl, cycloalkyl or aroyl, or R₁ and R₂ form a heterocyclic ring with the adjacent nitrogen atom;
X is O or S;
n is 1 or 2;
   and/or geometric isomers and/or stereoisomers and/or diastereomers and/or salts and/or hydrates and/or solvates and/or polymorphs thereof.

In certain embodiments, when R₁ and/or R₂ represent alkyl, the alkyl moiety is a substituted or unsubstituted saturated hydrocarbon radical which may be straight-chain or branched-chain and contains about 1 to about 6 carbon atoms (e.g., 1 to 4 carbon atoms), and is optionally substituted with one or more C₁₋₆ alkoxycarbonyl, aryl (e.g., phenyl) or (C₁₋₆ alkoxycarbonyl)-C₁₋₆ alkyl groups, or combinations thereof.

In additional embodiments, R₁ and R₂ form a heterocyclic ring with the adjacent nitrogen atom, which may be a saturated or unsaturated, optionally substituted, monocyclic or bicyclic ring, which may contain further heteroatoms selected from O, N, or S. For example, the heterocyclic ring can be pyrrolidine, piperazine, piperidine or morpholine.

In additional embodiments, when R₁ and/or R₂ represent alkenyl, the alkenyl moiety may have 2 to 7 carbon atoms and 1 to 3 double bonds.

In additional embodiments, when R₁ and/or R₂ represent aryl, the aryl moiety may be selected from an optionally substituted mono-, bi- or tricyclic aryl, such as, but not limited to, phenyl, naphthyl, fluorononyl, or anthraquinonyl group (e.g., phenyl or naphthyl). The aryl moiety may be substituted with one or more C₁₋₆ alkoxy, trifluoro-C₁₋₆ alkoxy, C₁₋₆ alkoxycarbonyl, C₁₋₆ alkanoyl, aryl, C₁₋₆ alkylthio, halogen, cyano groups or combinations thereof.

In additional embodiments, when R₁ and/or R₂ represent cycloalkyl, the cycloalkyl moiety may be selected from an optionally substituted mono-, bi- or tricyclic cycloalkyl group, such as cyclohexyl or adamantyl.

In additional embodiments, when R₁ and/or R₂ represent aroyl the aryl moiety therein is as defined above, e.g., phenyl.

In one embodiment, the compound of formula (I) is trans-1-{4-[2-[4-(2,3-dichlorophenyl)-piperazin-1-yl]-ethyl]-cyclohexyl}-3,3-dimethyl-urea, or a pharmaceutically acceptable salt thereof. For example, the compound of formula (I) is trans-1-{4-[2-[4-(2,3-dichlorophenyl)-piperazin-1-yl]-ethyl]-cyclohexyl}-3,3-dimethyl-urea hydrochloride.

In further embodiments, the present invention relates to pharmaceutical compositions comprising an NMDA receptor antagonist and trans-1-{4-[2-[4-(2,3-dichlorophenyl)-piperazin-1-yl]-ethyl]-cyclohexyl}-3,3-dimethyl-urea, or a pharmaceutically acceptable salt thereof (e.g., trans-1-{4-[2-[4-(2,3-dichlorophenyl)-piperazin-1-yl]-ethyl]-cyclohexyl}-3,3-dimethyl-urea hydrochloride).

In a further embodiment, the present invention relates to pharmaceutical compositions comprising a compound of formula (I), or a pharmaceutically acceptable salt thereof and memantine, or a pharmaceutically acceptable salt thereof (e.g., memantine hydrochloride).

In a further embodiment, the present invention relates to pharmaceutical compositions comprising trans-1-{4-[2-[4-(2,3-dichlorophenyl)-piperazin-1-yl]-ethyl]-cyclohexyl}-3,3-dimethyl-urea, or a pharmaceutically acceptable salt thereof and memantine, or a pharmaceutically acceptable salt thereof.

In another embodiment, the present invention relates to pharmaceutical compositions comprising trans-1-{4-[2-[4-(2,3-dichlorophenyl)-piperazin-1-yl]-ethyl]-cyclohexyl}-3,3-dimethyl-urea hydrochloride and memantine hydrochloride.

In another embodiment, the present invention relates to pharmaceutical compositions comprising trans-1-{4-[2-[4-(2,3-dichlorophenyl)-piperazin-1-yl]-ethyl]-cyclohexyl}-3,3-dimethyl-urea, or a pharmaceutically acceptable salt thereof, and neramexane, or a pharmaceutically acceptable salt thereof.

In another embodiment, the present invention relates to pharmaceutical compositions comprising trans-1-{4-[2-[4-(2,3-dichlorophenyl)-piperazin-1-yl]-ethyl]-cyclohexyl}-3,3-dimethyl-urea hydrochloride and neramexane mesylate.

In another embodiment, the present invention relates to pharmaceutical compositions comprising trans-1-{4-[2-[4-(2,3-dichlorophenyl)-piperazin-1-yl]-ethyl]-cyclohexyl}-3,3-dimethyl-urea hydrochloride and neramexane hydrochloride.

In another embodiment, the present invention relates to pharmaceutical compositions consisting essentially of an NMDA receptor antagonist and a compound of formula (I). For example, pharmaceutical compositions consisting essentially of an NMDA receptor antagonist and a compound of formula (I) include no additional pharmaceutically active ingredients, but may include additional pharmaceutical media known to one of ordinary skill in the art (e.g., carriers, additives, excipients etc.).

In another embodiment, the present invention relates to pharmaceutical compositions consisting essentially of cariprazine or a pharmaceutically acceptable salt thereof and memantine or a pharmaceutically acceptable salt thereof. For example, pharmaceutical compositions consisting essentially of cariprazine or a pharmaceutically acceptable salt thereof and memantine or a pharmaceutically acceptable salt thereof include no additional pharmaceutically active ingredients, but may include additional pharmaceutical media known to one of ordinary skill in the art (e.g., carriers, additives, excipients etc.). In another embodiment, the present invention relates to pharmaceutical compositions consisting essentially of cariprazine hydrochloride and memantine hydrochloride.

In a further aspect, the present invention relates to methods of treating a disorder selected from mania, bipolar disorder and schizophrenia wherein a patient is administered a pharmaceutical composition consisting essentially of an NMDA receptor antagonist and a compound of formula (I). In another embodiment, the present invention relates to methods of treating a disorder selected from mania, bipolar disorder and schizophrenia wherein a patient is administered a pharmaceutical composition consisting essentially of cariprazine or a pharmaceutically acceptable salt thereof and memantine or a pharmaceutically acceptable salt thereof. In other embodiments, the present invention relates to methods of treating a disorder selected from mania, bipolar disorder and schizophrenia wherein a patient is administered a pharmaceutical composition consisting essentially of cariprazine hydrochloride and memantine hydrochloride.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride salt, a hydrobromide salt or a mesylate salt.

Some of the compounds useful in the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. The use of such polymorphs is within the scope of the present invention.

Some of the compounds useful in the present invention can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates. The use of such solvates is within the scope of the present invention.

One of ordinary skill in the art will recognize that compounds useful in the present invention can exist in different tautomeric and geometrical isomeric forms. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, such as no more than 2%, for example no more than 1%.

The optical isomers can be obtained by resolution of the racemic mixtures of conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formula I can likewise be obtained by utilizing optically active starting materials in chiral synthesis processes under reaction conditions which do not cause racemization.

In addition, one of ordinary skill in the art will recognize that the compounds useful in the present invention can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] (2000), 110 pp. CAN 133:68895 AN 2000:473538 CAPLUS; Kabalka, George W.; Varma, Rajender S. The synthesis of radiolabeled compounds via organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64 (1-2), 9-32. CODEN: JRACBN ISSN:0022-4081, CAN 95:76229 AN 1981:476229 CAPLUS.

### Dosage Forms

Numerous standard references are available that describe procedures for preparing various formulations suitable for administering the compounds of the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage forms is closely related to the therapeutic amounts of the compounds or compositions which are desirable and efficacious for the given treatment application.

Suitable dosage forms include, but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterille administration, and other dosage forms for systemic delivery of active ingredients. Formulations suitable for oral administration are preferred.

To prepare such pharmaceutical dosage forms, the active ingredient(s) is/are typically mixed with a pharmaceutical carrier of conventional pharmaceutical compounding techniques. The carrier may take a wide variety of forms depending on the form of preparation desired for administration.

In preparing the compositions in oral dosage form, any of the usual pharmaceutical media known to one of ordinary skill in the art may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each comprising a predetermined amount of the active ingredient as a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery or delivery directly to the CNS. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The formulations of the present invention can have immediate release, sustained release, delayed-onset release or any other release profile known to one skilled in the art.

### Dosages

In some embodiments, the NMDA receptor antagonist (such as memantine, or a pharmaceutically acceptable salt thereof, e.g., memantine hydrochloride) may be administered in doses ranging from about 0.1 mg to about 60 mg/day. In some embodiments, memantine, or a pharmaceutically acceptable salt thereof, may be administered in doses ranging from about 1 mg to about 60 mg/day. In other embodiments, memantine, or a pharmaceutically acceptable salt thereof, may be administered in doses ranging from about 5 mg to about 20 mg/day, e.g., 10 mg/day, 20 mg/day.

In additional embodiments, neramexane, or a pharmaceutically acceptable salt thereof (e.g., neramexane hydrochloride, neramexane mesylate) may be administered in doses ranging from 0.1-100 mg/day. In some embodiments, neramexane, or a pharmaceutically acceptable salt thereof, may be administered in doses ranging from about 10-100 mg/day. In other embodiments, neramexane, or a pharmaceutically acceptable salt thereof, may be administered in doses ranging from about 25-75 mg/day. In yet other embodiments, neramexane, or a pharmaceutically acceptable salt thereof, is administered in doses ranging from about 10-20 mg/day.

The dose of the active agents administered in the methods described herein is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, and seriousness of the disease. The appropriate dose and dosage times under certain conditions can be determined by the test based on the above-described indices but may be refined and ultimately decided of the judgment of the practitioner and each patient's circumstances (age, general condition, severity of symptoms, sex, etc.) of standard clinical techniques.

For combination treatments, the active ingredients can normally be administered in a combined daily dosage regimen (for an adult patient) of, for example, between about 0.1 mg and about 500 mg, such as between about 10 mg and about 400 mg, e.g. between about 10 mg and about 250 mg or an intravenous, subcutaneous, or intramuscular dose of between about 0.1 mg and about 100 mg, such as between about 0.1 mg and about 50 mg, e.g. between about 1 mg and about 25 mg.

In certain embodiments, the pharmaceutical compositions disclosed herein include about 0.05 mg, about 0.1 mg, about 0.2 mg, about 0.3 mg, about 0.4 mg, about 0.5 mg, about 0.75 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg or about 15 mg of a compound of formula I, or pharmaceutically acceptable salt thereof (e.g., trans-1-{4-[2-[4-(2,3-dichlorophenyl)-piperazin-1-yl]-ethyl]-cyclohexyl}-3,3-dimethyl-urea hydrochloride). For example, the pharmaceutical compositions include about 0.1 mg, about 0.25 mg, about 0.5 mg, about 1 mg, about 1.5 mg, about 2 mg, about 2.5 mg, about 3 mg, about 4.5 mg, about 5 mg, about 6 mg, about 7.5 mg, about 9 mg, about 12.5 mg, or about 15 mg of a compound of formula I, or pharmaceutically acceptable salt thereof (e.g., trans-1-{4-[2-[4-(2,3-dichlorophenyl)-piperazin-1-yl]-ethyl]-cyclohexyl}-3,3-dimethyl-urea hydrochloride). In certain embodiments, the pharmaceutical compositions include about 1.5 mg, about 3 mg or about 4.5 mg of a compound of formula I, or pharmaceutically acceptable salt thereof (e.g., trans-1-{4-[2-[4-(2,3-dichlorophenyl)-piperazin-1-yl]-ethyl]-cyclohexyl}-3,3-dimethyl-urea hydrochloride).

In yet further embodiments, the compound of formula I, or a pharmaceutically acceptable salt thereof is present in the composition in an amount, which ranges between any two of these dosage amounts (e.g., between about 0.5 mg and about 12 mg, between about 1.5 mg and about 4.5 mg, between about 6 mg and about 12 mg).

In a further embodiment, the pharmaceutical composition contains about 1 mg, about 1.5 mg, about 2.0 mg, about 2.5 mg, about 4 mg, about 5 mg, about 10 mg, about 15 mg, about 20 mg, about 25 mg or about 30 mg of an NMDA receptor antagonist, e.g., memantine, or a pharmaceutically acceptable salt thereof (e.g., memantine hydrochloride) or neramexane, or a pharmaceutically acceptable salt thereof (e.g., neramexane hydrochloride, neramexane mesylate). For example, the pharmaceutical composition contains about 5 mg, about 10 mg or about 20 mg of memantine, or a pharmaceutically acceptable salt thereof (e.g., memantine hydrochloride).

Unitary dosage forms comprising memantine hydrochloride and trans-1-{4-[2-[4-(2,3-dichlorophenyl)-piperazin-1-yl]-ethyl]-cyclohexyl}-3,3-dimethyl-urea hydrochloride may be formulated so that the memantine hydrochloride and trans-1-{4-[2-[4-(2,3-dichlorophenyl)-piperazin-1-yl]-ethyl]-cyclohexyl}-3,3-dimethyl-urea hydrochloride are not in contact with one another.

Unitary dosage forms comprising neramexane hydrochloride (or neramexane mesylate) and trans-1-{4-[2-[4-(2,3-dichlorophenyl)-piperazin-1-yl]-ethyl]-cyclohexyl}-3,3-dimethyl-urea hydrochloride may be formulated so that the neramexane hydrochloride (or neramexane mesylate) and trans-1-{4-[2-[4-(2,3-dichlorophenyl)-piperazin-1-yl]-ethyl]-cyclohexyl}-3,3-dimethyl-urea hydrochloride are not in contact with one another.

The desired dose may be administered as one or more daily sub dose(s) administered at appropriate time intervals throughout the day, or alternatively, in a single dose, for example, for morning or evening administration. For example, the daily dosage may be divided into one, into two, into three, or into four divided daily doses.

The duration of the treatment may be decades, years, months, weeks, or days, as long as the benefits persist.

The compound of formula (I) and the NMDA receptor antagonist may be administered concurrently (either as separate dosage forms or in a combined dosage form) or the compound of formula (I) may be administered prior to or subsequent to administration of the NMDA receptor antagonist.

In another embodiment, both compounds are administered in sub-optimal or sub-threshold doses and the combination results in a synergistic therapeutic effect.

### DEFINITIONS

The term “memantine” as used herein includes 1-amino-3,5-dimethyl adamantane and pharmaceutically acceptable salts thereof. Preferred pharmaceutically acceptable salts of memantine include, but are not limited to, memantine hydrochloride. The term “memantine” also includes polymorphs, hydrates, solvates, and amorphous forms of memantine and its pharmaceutically acceptable salts.

The term “neramexane” as used herein includes 1-amino-1,3,3,5,5-pentamethylcyclohexane and pharmaceutically acceptable salts thereof. Preferred pharmaceutically acceptable salts of neramexane include, but are not limited to, neramexane mesylate and neramexane hydrochloride.

The term “sub-threshold” refers to the amount of an active ingredient inadequate to produce a response, e.g., an amount below the minimum effective amount when the active ingredient is used as monotherapy.

The term “sub-optimal” in the same context means an amount of an active ingredient that produces a response but not to its full extent, which would be achieved with a higher amount.

The term “synergistic” refers to the combined effect of administering two therapeutic compounds where the overall response is greater than the sum of the two individual effects. The term synergy also refers to the combined effect of administering an amount of one compound that, when administered as monotherapy, produces no measurable response but, when administered in combination with another therapeutic compound, produces an overall response that is greater than that produced by the second compound alone.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

The term “schizophrenia” is intended to include the group of mental disorders characterized by disruptions in thinking and perception, and includes schizophrenia (and all its subtypes; paranoid, catatonic, disorganized, residual, undifferentiated) and other psychotic disorders (as per Diagnostic and Statistical Manual for Mental Disorders, Fourth Edition, Washington, D.C (1994): American Psychiatric Association, or The ICD-10 Classification of Mental and Behavioural Disorders: Clinical Descriptions and Diagnostic Guidelines, Geneva (1992): World Health Organization) such as schizophreniform and schizoaffective disorders, brief psychotic disorder, etc. For example, the term “schizophrenia” is intended to include schizoaffective, shcizophreniform disorder, depression in schizophrenia, suicidality in schizophrenia and treatment resistant schizophrenia. In addition, the term “schizophrenia” is intended to include the symptoms associated with schizophrenia including the cognitive symptoms of schizophrenia, cognitive impairment associated with schizophrenia, cognitive deficits associated with schizophrenia, affective symptoms of schizophrenia, positive symptoms of schizophrenia, and negative symptoms of schizophrenia.

In a clinical evaluation, schizophrenia is commonly marked by “positive symptoms” such as hallucinations (especially auditory hallucination which are usually experienced as voices), disorganized thought processes and delusions as well as “negative symptoms” which include affective flattening, alogia, avolition, and anhedonia.

The term “the negative symptoms of schizophrenia” refer to a class of symptoms of schizophrenia which can be considered to reflect a ‘loss’ in functional, directed thought or activity. Negative symptoms of schizophrenia are well known in the art, and include affective flattening (characterized by, for example, an immobile and/or unresponsive facial expression, poor eye contact and reduced body language), alogia (‘poverty of speech’ or brief, laconic and/or empty replies), avolition (characterized by a reduced or absent ability to initiate and carry out goal-directed activities), anhedonia (loss of interest or pleasure), asocialty (reduced social drive and interaction), apathy and other negative symptoms known to those of skill in the art. The negative symptoms of schizophrenia may be assessed using any methodology known in the art including, but not limited to, the Brief Psychiatric Rating Scale (BPRS), and the Positive and Negative Symptom Scale (PANSS). The BPRS and PANSS have subscales or factors that can be used to measure negative symptoms. Other scales have been designed to address specifically negative symptoms: For example the Scale for the Assessment of Negative Symptoms (SANS), the Negative Symptoms Assessment (NSA) and the Schedule for the Deficit Syndrome (SDS). Subscales of the BPRS and PANSS may also be used to assess positive symptoms, although methods for specifically assessing positive symptoms are also available (e.g., the Scale for the Assessment of Positive Symptoms, or SAPS).

The terms “cognitive impairment associated with schizophrenia” and “cognitive deficits associated with schizophrenia” refers to cognitive deficits in schizophrenia patients. Cognitive impairment in schizophrenia is a core feature of the illness (e.g. not a result of treatment or clinical symptoms). Cognitive deficits include, but are not limited to deficits of attention/vigilance, working memory, verbal learning and memory, visuospatial memory, reasoning/problem solving and social cognition. There are numerous neuropsychological tests used to measure cognitive deficits in schizophrenia, such as the Wisconsin Card Sorting Test (WCST).

The term “autism” refers to an individual demonstrating any one or all of the symptoms and characteristics associated with autism. Such individual may fit particular diagnostic criteria, such as Autistic Disorder, Asperger's Disorder, Atypical Autism or Pervasive Developmental Disorder, NOS (not otherwise specified), Rett's Disorder or Childhood Disintegrative Disorder, or the broader autism phenotype disorder or such individual may not fit a discrete diagnostic category at all. Due to the many presentations of the disease called autism, the present invention will use the term “autism” to refer to all of the above disorders.

The terms “treat,” “treatment,” and “treating” refer to one or more of the following: relieving or alleviating at least one symptom of a disorder in a subject; relieving or alleviating the intensity and/or duration of a manifestation of a disorder experienced by a subject; and arresting, delaying the onset (e.g., the period prior to clinical manifestation of a disorder) and/or reducing the risk of developing or worsening a disorder.

The term “mood disorder” as used herein includes the mood disorders specified in the DSM-IV-TR, including, but not limited to, major depressive disorder, bipolar disorder, unipolar depression, dysthymia, cyclothymia, seasonal affective disorder, and post-partum depression. The term mood disorder also refers to secondary depression resulting from a systemic or neurological disease. Examples of neurologic diseases include multiple sclerosis, Parkinson's disease, Alzheimer's disease, head trauma, cerebral tumors, post-stroke, early dementia, and sleep apnea, while systemic diseases include, but are not limited to infections, endocrine disorders, collagen vascular diseases, nutritional deficiencies and neoplastic disease. Secondary depression is also common in post-myocardial infarct patients, who exhibit a mortality three times that of non-depressed post-myocardial patients.

The term “NMDA receptor antagonists” is used to refer to drugs that can attenuate NMDA receptor-mediated neuronal activity. Preferred NMDA receptor antagonists are 1-aminocyclohexane derivatives and analogs, including aminoadamantanes such as memantine and neramexane and pharmaceutically acceptable salts thereof. An NMDA “functional antagonist” is any compound which possesses pharmaceutically efficacious properties in humans, and which reduces excessive activity at NMDA responsive cation channels. “Functional” antagonists of the invention include compounds which are partial agonists of the strychnine-insensitive glycine binding site, as well as competitive and non-competitive antagonists at the NMDA receptor complex at other binding sites. This term also includes agents that modify the NMDA receptor in any way.

An NMDA “competitive antagonist” is a compound possessing competitive antagonist properties when compared with endogenous neurotransmitters glutamate and aspartate.

An NMDA “non-competitive antagonist” is a compound that reduces activity at NMDA-gated cation channels at loci other than the strychnine-insensitive glycine binding site or the NMDA binding site. An example of such site is within the cation channel, such as memantine. Non-competitive antagonists are preferred.

An “effective amount” means the amount of a composition of the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical composition that is sufficient to result in a desired activity upon administration to a mammal in need thereof. As used herein with respect to the pharmaceutical compositions comprising an NMDA receptor antagonist, e.g., memantine hydrochloride, and compound of Formula (I), e.g., trans-1-{4-[2-[4-(2,3-dichlorophenyl)-piperazin-1-yl]-ethyl]-cyclohexyl}-3,3-dimethyl-urea hydrochloride, the term “therapeutically effective amount/dose” refers to the amount/dose of each compound that, when combined, is sufficient to produce an effective response upon administration to a mammal.

A subject or patient in whom administration of the therapeutic compound is an effective therapeutic regimen for a disease or disorder is preferably a human, but can be any animal, including a laboratory animal in the context of a trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

A multicenter, open-label study to evaluate the efficacy and tolerability of memantine hydrochloride in the acute treatment of hospitalized patients with bipolar I disorder experiencing a manic or mixed episode was conducted.

The entrance criteria for the patients included:

Hospitalized adult inpatients, with bipolar I disorder by DSM-IV-TR criteria
Manic or mixed episode: ≧20 on the Young Mania Rating Scale (YMRS), with and without psychotic features;
Diagnosis based on clinical evaluation and confirmed using the Structured Clinical Interview for DSM Disorders.

The following comorbid psychiatric diagnoses were allowed to enroll: Attention deficit hyperactivity disorder [ADHD], conduct disorder, obsessive-compulsive disorder, anxiety disorders, and substance abuse. Additional psychotropic medications were not allowed.

Patients were assigned to 21-days of treatment in one of three groups:

Group 1, 20 mg memantine hydrochloride per day (range 20-30 mg memantine per day);

Group 2, 30 mg memantine hydrochloride per day (range 20-40 mg memantine per day);

Group 3, 40 mg memantine hydrochloride per day (range 30-50 mg memantine per day).

One increase/decrease of 10 mg memantine hydrochloride per day (minimum 20 mg memantine per day) was permitted during the first 8 days of treatment for patients with dose-limiting adverse events (AEs) or insufficient therapeutic response. Dose modification was continued to study completion or early termination.

One blood sample (˜7 mL) per patient was collected immediately before dosing on Day 13 (Day 21 for those with dosing modification) to determine the memantine hydrochloride trough plasma concentration following multiple dosing

Efficacy measures included the Young Mania Rating Scale (YMRS) and the Mania Rating Scale (MRS) (≧50% reduction in total score from baseline). The change from baseline was also assessed using the Positive and Negative Syndrome Scale (PANSS) in patients with psychiatric symptoms, Positive and Negative Syndrome Scale-Excited Component (PANSS-EC), Clinical Global Impression Severity (CGI-S) and Improvement (CGI-I) scores, and the change from baseline in the Montgomery Asberg Depression Rating Scale (MADRS).

A total of 35 patients were enrolled; 33 received at least one dose of memantine hydrochloride and had at least one post baseline assessment using YMRS. The mean duration of treatment was 16.1 days (Group I), 14.7 days (Group 2) and 17.3 days (Group 3). The mean compliance with the memantine hydrochloride treatment was ≧90%.

Table 1 shows the efficacy as measured from baseline to Day 21 using the Young Mania Rating Scale and the Mania Rating Scale in the intent-to-treat population.

TABLE 1

| | | Group 1 | | Group 2 | | Group 3 | |
|---|---|---|---|---|---|---|---|
| | | | Mean ± | | Mean ± | | Mean ± |
| Parameter | Time | n | SEM | n | SEM | n | SEM |
| YMRS | Baseline | 12 | 26.8 ± 1.3 | 11 | 25.4 ± 1.1 | 10 | 26.4 ± 1.3 |
| Total | Change at | 12 | −9.5 ± 2.8 | 11 | −6.4 ± 3.8 | 10 | −7.9 ± 2.4 |
| Score | Day 21 (LOCF) | | | | | | |
| | Change at | 6 | −17.0 ± 1.9 | 6 | −15.5 ± 1.5 | 8 | −10 ± 2.3 |
| | Day 21 (OC) | | | | | | |
| MRS Total | Baseline | 12 | 25.3 ± 1.1 | 11 | 25.3 ± 1.4 | 10 | 26.4 ± 1.2 |
| Score | Change at | 12 | −9.8 ± 2.2 | 11 | −7.4 ± 2.6 | 10 | −7.2 ± 2.0 |
| | Day 21 (LOCF) | | | | | | |
| | Change at | 6 | −15.3 ± 1.7 | 6 | −13.0 ± 2.1 | 8 | −9.3 ± 1.8 |
| | Day 21 (OC) | | | | | | |

LOCF = last observation carried forward;
OC = observed cases.

Table 2 shows the changes associated with memantine as measured by other efficacy parameters (last observation carried forward) in the intent-to-treat population.

TABLE 2

| | | Group 1 | Group 2 | Group 3 |
|---|---|---|---|---|
| | | (n = 12) | (n = 11) | (n = 10) |
| | | Mean ± | Mean ± | Mean ± |
| Parameter | Time | SEM | SEM | SEM |
| PANSS Total | Baseline | 67.8 ± 4.8 | 63.2 ± 3.7 | 77.4 ± 2.5 |
| Score | Change at Day 21 | −5.1 ± 3.9 | −1.5 ± 5.8 | −7.7 ± 3.8 |
| PANSS-EC Score | Baseline | 14.0 ± 0.6 | 13.5 ± 1.0 | 14.5 ± 0.9 |
| | Change at Day 21 | −0.7 ± 1.5 | −0.5 ± 1.8 | −0.7 ± 1.4 |
| CGI-S Rating | Baseline | 4.4 ± 0.2 | 4.4 ± 0.2 | 4.3 ± 0.2 |
| | Change at Day 21 | −0.8 ± 0.3 | −0.7 ± 0.4 | −0.6 ± 0.3 |
| CGI-I Rating | Day 21 | 3.3 ± 0.5 | 3.6 ± 0.5 | 2.9 ± 0.5 |
| MADRS Total | Baseline | 14.3 ± 1.8 | 9.5 ± 1.0 | 13.8 ± 1.3 |
| Score | Change at Day 21 | −3.4 ± 1.8 | 0.0 ± 2.7 | −3.3 ± 1.7 |
| YMRS Re- | Day 21 | 6 (50.0) | 5 (45.5) | 3 (30.0) |
| sponse* n (%) | | | | |
| MRS Re- | Day 21 | 6 (50.0) | 3 (27.3) | 1 (10.0) |
| sponse* n (%) | | | | |

*Response defined as ≧50% reduction in total score from baseline.
SEM = standard error of the mean

The results show that memantine hydrochloride is surprisingly effective in the treatment of acute mania associated with bipolar disorder.

### Example 2

### Dual-Probe Microdialysis Analysis of Acetylcholine, Dopamine and Serotonin in the Frontal Cortex of Freely-Moving Rats

A dual-probe microdialysis approach will be used to determine the effects of oral administration of cariprazine hydrochloride (trans-4-{2-[4-(2,3-dichlorophenyl)-piperazin-1-yl]-ethyl}-N,N-dimethylcarbamoyl-cyclohexylamine hydrochloride), memantine hydrochloride, and combinations thereof on the extracellular concentrations of acetylcholine (ACh), dopamine (DA) and serotonin (5-HT) in the frontal cortex of freely-moving rats.

### Materials and Methods

### Animals and Environment

Experiments will be carried out in male Sprague-Dawley rats (250-350 g body weight). Animals will be housed in groups of six on a 12 h/12 h light/dark cycle (lights on at 07.30 h), at an ambient temperature of 21±2° C. and 55±20% humidity. Food and water will be available ad libitum. Animals will be allowed to acclimatize to these conditions for at least 5 days prior to the study.

### Microdialysis Protocol

Rats will be anaesthetised with isoflurane (5% to induce, 2% to maintain) in an O₂/N₂O (1 litre/min each) mixture delivered via an anaesthetic unit. Concentric microdialysis probes with 2 mm exposed Hospal membrane tip (CMA) will be stereotaxically implanted bilaterally into the prefrontal cortex using coordinates taken from the stereotaxic atlas of Paxinos and Watson (Paxinos G, Watson C. The Rat Brain in Stereotaxic Coordinates, 2^{nd} Edition, London: Academic Press, 1986). The upper incisor bar will be set at 3.3 mm below the interaural line so that the skull surface between bregma and lambda will be horizontal. The co-ordinates for each probe will be identical from bregma except for the lateral measurement as one probe will be placed to the left of the midline and the other to the right. Samples will be measured from the same side in each rat (e.g. left probe will measure ACh and right probe will measure DA and 5-HT). Additional burr holes will be made for skull screws (stainless steel) and the probes will be secured using dental cement. Following surgery, the animals will be individually housed in circular chambers (dimensions 450 mm internal diameter, 320 mm wall height) with the microdialysis probes connected to a liquid swivel and a counter-balanced arm to allow unrestricted movement. The rats will be allowed a recovery period of at least 16 h with food and water available ad libitum. During this time the probes will be continuously perfused with an artificial cerebrospinal fluid (aCSF) at a flow rate of 1.2 μl/min. The perfusate for one probe will contain neostigmine, a cholinesterase inhibitor to prevent the breakdown of ACh in these samples.

The experiment will be performed the day following surgery. Dialysate samples will be collected every 20 min from 80 min before drug administration until 240 min after drug administration (16 samples in total from each probe; 4 pre-drug and 12 post-drug). Dialysate samples will be collected into Eppendorf tubes containing perchloric acid (samples for measurement of DA and 5-HT only) to prevent oxidation and all samples frozen for storage at −80° C. until analysis. The HPLC analysis of DA and 5-HT in one set of samples and of ACh in the second set will be conducted over the remainder of the week following experimentation.

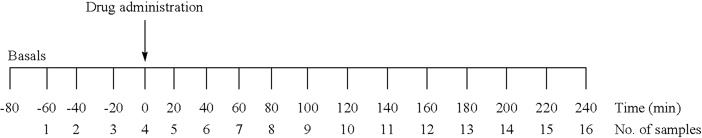

The study will progress at a rate of approximately 6 rats per week. The experiment will include a vehicle-treated control group and will therefore consist of 8 different groups (A-H). Final group sizes of 7 will be employed. A summary of the proposed treatment groups is shown below:

| Group | Treatment | n |
|---|---|---|
| A | Vehicle (po) | 7 |
| B | Memantine hydrochloride (1.0 mg/kg s.c.) | 7 |
| C | trans-4-{2-[4-(2,3-dichlorophenyl)-piperazin-1-yl]-ethyl}-N,N- | 7 |
| | dimethylcarbamoyl-cyclohexylamine hydrochloride (0.1 mg/kg po) | |
| D | trans-4-{2-[4-(2,3-dichlorophenyl)-piperazin-1-yl]-ethyl}-N,N- | 7 |
| | dimethylcarbamoyl-cyclohexylamine hydrochloride (0.3 mg/kg po) | |
| E | trans-4-{2-[4-(2,3-dichlorophenyl)-piperazin-1-yl]-ethyl}-N,N- | 7 |
| | dimethylcarbamoyl-cyclohexylamine hydrochloride (1 mg/kg po) | |
| F | trans-4-{2-[4-(2,3-dichlorophenyl)-piperazin-1-yl]-ethyl}-N,N- | 7 |
| | dimethylcarbamoyl-cyclohexylamine hydrochloride (0.1 mg/kg po) + | |
| | Memantine hydrochloride (1.0 mg/kg s.c.) | |
| G | trans-4-{2-[4-(2,3-dichlorophenyl)-piperazin-1-yl]-ethyl}-N,N- | 7 |
| | dimethylcarbamoyl-cyclohexylamine hydrochloride (0.3 mg/kg po) + | |
| | Memantine hydrochloride (1.0 mg/kg s.c.) | |
| H | trans-4-{2-[4-(2,3-dichlorophenyl)-piperazin-1-yl]-ethyl}-N,N- | 7 |
| | dimethylcarbamoyl-cyclohexylamine hydrochloride (1 mg/kg po) + | |
| | Memantine hydrochloride (1.0 mg/kg s.c.) | |

At the end of the experiments, the animals will be sacrificed, their brains removed, sectioned (using a vibratome) and mounted on slides. Probe placements will be visualized and verified using a stereotaxic atlas (Paxinos and Watson, 1986). Only data from rats with correctly positioned probes will be used in the analysis.

### Compounds

All test compounds will be dissolved in a suitable vehicle and administered orally via gavage using a suitable dose volume. The solutions will be made up fresh each day within 1-2 h of dosing. All compound doses will be expressed as the free base using the appropriate salt:free base factor.

### Date and Statistical Analysis

Statistical analysis will be performed by a qualified statistician. In all experiments, the average of the 4 pre-drug administration values will be used as a measure of basal levels. Results will be expressed as means±S.E.M. Statistical analysis of post-treatment responses will be typically by one-way analysis of covariance (ANCOVA) on log transformed data for each time point with group as a factor and baseline as a covariate. Data will be examined assuming normal distribution and equal variances and any extreme values and any which are z<−3 or z<3 (where z=residual from the statistical model using log-transformed data) will not included. Statistical comparisons between the different treatment groups will be made by suitable post hoc multiple comparisons tests. A P value of <0.05 will be considered statistically significant.

### Example 3

### The Effect of a Combination of Cariprazine Hydrochloride and Memantine Hydrochloride in an Animal Model for Mania

Cariprazine hydrochloride (trans-4-{2-[4-(2,3-dichlorophenyl)-piperazin-1-yl]-ethyl}-N,N-dimethylcarbamoyl-cyclohexylamine hydrochloride) is expected to be an effective antimanic agent in bipolar illness. Memantine hydrochloride may synergistically potentiate the antimanic effect of trans-4-{2-[4-(2,3-dichlorophenyl)-piperazin-1-yl]-ethyl}-N,N-dimethylcarbamoyl-cyclohexylamine hydrochloride. These predictions will be tested utilizing the ouabain animal model of mania and the hippocampal slice model.

### Proposed Studies

### Animal Model Studies

The ouabain animal model for mania is based on open field behavior after an ICV injection of 5 μL of 10⁻⁵M ouabain dissolved in artificial cerebrospinal fluid (aCSF). This dose of ICV ouabain causes motoric hyperactivity which is normalized by prior administration of lithium (at ‘therapeutic levels’). Activity is observed over a thirty minute period in an open field. The open field is a large, open 86×86 cm arena with 16 squares (21.5×21.5 cm) marked on the floor. As described in El-Mallakh R S et al., Bipolar Disorders, 5:362-365, 2003, ouabain treated animals had an increase in normal exploratory activity (sham 53.1±SD 50.0; ouabain treated 258.7±316.6 squares traversed, Fisher PLSD=130.8, P<0.5), and were observed to be more aggressive when handled. Lithium alone has no effect (79.3±53.3). Lithium pretreatment normalizes this behavior, i.e., prevents ouabain-induced hyperlocomotion (75.8±94.7, Fisher PLSD=147.7, P<0.05).

For the proposed studies, one group will include the evaluation of memantine alone (1.0 mg/kg s.c.) which will be administered immediately after ICV ouabain injection. In order to evaluate the effect of the combination, trans-4-{2-[4-(2,3-dichlorophenyl)-piperazin-1-yl]-ethyl}-N,N-dimethylcarbamoyl-cyclohexylamine hydrochloride (4 doses—0.06, 0.25, 0.5 or 1.0 mg/kg i.p.) will be administered in the absence or presence of a fixed dose of memantine hydrochloride (1.0 mg/kg s.c.) immediately after ICV ouabain injection. Open field activity will be examined one hour after drug administration.

In addition, lithium will be tested as a positive control. Lithium will be administered IP at 6.75 mEq/kg immediately after ICV ouabain injection. Open field activity will be examined 18 hours after lithium administration since peak brain lithium levels are achieved at this time.

Two other groups of animals will be given vehicle IP immediately after ICV ouabain injection and observed at one hour and 24 hours, respectively, after vehicle administration. Artificial cerebrospinal fluid (aCSF) ICV controls will also be examined for each drug dosage.

After the behavioral observations conducted immediately following the initial treatment, the animals will be placed in cages where they will receive (i) memantine hydrochloride (1.0 mg/kg s.c.), (ii) trans-4-{2-[4-(2,3-dichlorophenyl)-piperazin-1-yl]-ethyl}-N,N-dimethylcarbamoyl-cyclohexylamine hydrochloride at 4 doses (0.06, 0.25, 0.5 and 1.0 mg/kg, i.p.) in the presence or absence of memantine hydrochloride (1.0 mg/kg, s.c.) or (iii) lithium at 1.994 g/kg (in the food) for an additional 7 days when behavioral observations will again be made. This will provide information about the chronic effects of these drugs.

Animals. Male Sprague-Dawley rats (225-275 gm) will be housed individually with food and water available ad libitum under a 12:12 hr light:dark cycle, and allowed one week to acclimate to the facility.

Behavioral Testing. All behavioral data will be collected during the light hours. Open field activity will be recorded in a large, open 86×86 cm arena with 16 squares (21.5×21.5 cm) marked on the floor. Activity will be quantified by a trained observer as the number of squares traversed in 30 min.

Time Line. Behavioral testing will be performed on days 0, 7, and 14. Day 0 will be used to acclimatize the animals to the testing environment. ICV cannulae will be surgically placed on day 3. Primary outcome behavioral testing will be performed on day 7 after IP administration of the test compound (following ICV administration of ouabain or aCSF), and on day 8 after IP. administration of lithium. Behavioral testing will again be performed on day 14, after one week of daily administration of the test drugs. Animals will be sacrificed after completion of behavioral testing on day 14, and plasma collected for memantine, trans-4-{2-[4-(2,3-dichlorophenyl)-piperazin-1-yl]-ethyl}-N,N-dimethylcarbamoyl-cyclohexylamine hydrochloride and lithium level determinations. Drug levels will also be determined after single administration of each drug. The outline of the proposed animal model studies is shown below:

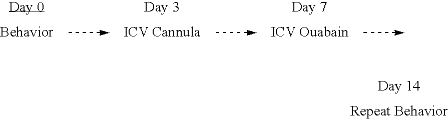

Cannulae. ICV cannulae will be surgically placed in the left lateral ventricle as previously described (El-Mallakh R S et al., Biol Psychiatry, 1995, 19:955-962; Changaris D G et al., Regul Pept, 1988, 20:273-280. Briefly, following anesthesia with intramuscular ketamine (90.0 mg/kg) and acepromazine (0.91 mg/kg) cannulae will be inserted to 3.5 mm through a no. 60 hole drilled in the dorsal aspect of the skull 2.5 mm lateral and 1 mm caudal to bregma using a stereotactic setup. Cannulae will be anchored with dental cement and plugged with a wire. At the end of the experiments, all animals will be sacrificed following halothane anesthesia and the brain dissected and examined visually to ensure lack of injury.

Drug Administration. Both lithium and trans-4-{2-[4-(2,3-dichlorophenyl)-piperazin-1-yl]-ethyl}-N,N-dimethylcarbamoyl-cyclohexylamine hydrochloride will be administered intraperitoneally and memantine subcutaneously at different phases of the study. Chronic memantine hydrochloride (s.c.), trans-4-{2-[4-(2,3-dichlorophenyl)-piperazin-1-yl]-ethyl}-N,N-dimethylcarbamoyl-cyclohexylamine hydrochloride (i.p.)±memantine hydrochloride (s.c.) will be administered for 7 days after ICV ouabain administration. Four doses of trans-4-{2-[4-(2,3-dichlorophenyl)-piperazin-1-yl]-ethyl}-N,N-dimethylcarbamoyl-cyclohexylamine hydrochloride will be tested (0.06, 0.25, 0.5 and 1 mg/kg, i.p.) in the presence or absence of a fixed dose of memantine hydrochloride (1.0 mg/kg, s.c.). Only a single oral dose of lithium (1.994 g/kg in the food) will be examined as a positive control. Similarly, trans-4-{2-[4-(2,3-dichlorophenyl)-piperazin-1-yl]-ethyl}-N,N-dimethylcarbamoyl-cyclohexylamine hydrochloride will be administered acutely at four doses (0.06, 0.25, 0.5 and 1 mg/kg, i.p.) in the presence or absence of a fixed dose of memantine (1.0 mg/kg, s.c.). Lithium will be administered at 6.75 mEq/kg i.p. Ouabain will be dissolved in aCSF at 10⁻⁵M and administered in a 5 μL volume through the ICV cannulae. Control animals will receive 5 μL of aCSF. Blood will be collected after decapitation at both days 7 and 14, and serum will be isolated for determination of trans-4-{2-[4-(2,3-dichlorophenyl)-piperazin-1-yl]-ethyl}-N,N-dimethylcarbamoyl-cyclohexylamine hydrochloride, memantine hydrochloride and lithium levels.

Treatment Groups. There will be 21 treatment groups: Group 1) Control (receiving drinking water and aCSF per ICV); Groups 2, 3, 4, 5) trans-4-{2-[4-(2,3-dichlorophenyl)-piperazin-1-yl]-ethyl}-N,N-dimethylcarbamoyl-cyclohexylamine hydrochloride alone treated rats at 4 doses (0.06, 0.25, 0.5 and 1 mg/kg, i.p.); Group 5) memantine hydrochloride alone at a fixed dose (1.0 mg/kg, s.c.); Groups 6, 7, 8, 9) trans-4-{2-[4-(2,3-dichlorophenyl)-piperazin-1-yl]-ethyl}-N,N-dimethylcarbamoyl-cyclohexylamine hydrochloride at 4 doses (0.06, 0.25, 0.5 and 1 mg/kg) in combination with a fixed dose of memantine hydrochloride (1.0 mg/kg); Group 10) lithium alone at one dose (6.75 mEq/kg i.p.); Group 11) ICV ouabain (10⁻⁵ M); Group 12) memantine hydrochloride (1.0 mg/kg, s.c.) co-administered with ICV ouabain (10⁻⁵ M); Groups 13, 14, 15, 16) trans-4-{2-[4-(2,3-dichlorophenyl)-piperazin-1-yl]-ethyl}-N,N-dimethylcarbamoyl-cyclohexylamine hydrochloride at 4 doses (0.06, 0.25, 0.5 and 1 mg/kg) co-administered with ICV ouabain (10⁻⁵ M); Groups 17, 18, 19, 20) trans-4-{2-[4-(2,3-dichlorophenyl)-piperazin-1-yl]-ethyl}-N,N-dimethylcarbamoyl-cyclohexylamine hydrochloride at 4 doses (0.06, 0.25, 0.5 and 1 mg/kg) in combination with a fixed dose of memantine hydrochloride (1.0 mg/kg) and co-administered with ICV ouabain (10⁻⁵ M); Group 21) lithium (6.75 mEq/kg i.p.) co-administered with ICV ouabain (10⁻⁵ M).

Data Analysis. ANOVA with post hoc Fisher PLSD tests will be used. A probability level of <0.05 will be held as significant. 10-15 animals per group will be used.

### Hippocampal Slice Studies

See, e.g., El-Mallakh R S, Schurr A, Payne R S, Li R, Journal of Psychiatric Research 34:115-120, 2000.

Twenty to thirty 400 μm thick hippocampal slices will be prepared from each hippocampus. The experiment will be conducted in a dual linear-flow incubation/recording chamber. The slices will be bathed in aCSF and supplied with a humidified gas mixture of 95% O₂ and 5% CO₂ at a temperature of 34° C. Extracellular recordings from the stratum pyramidale of the CA1 region will be made with a borosilicate micropipette filled with aCSF connected to a 2 channel preamplifier. Evoked cellular responses to a stimulus pulse (at an amplitude twice the threshold) delivered to the Schaffer collaterals will be evaluated. Only evoked responses will be measured as they are more uniform and can be compared across experiments. Spontaneous hippocampal activity is not recorded because it is not consistent across slices. Ouabain will be infused at 3.3 μM, a concentration that is known to induce cycling. Memantine hydrochloride will be studied alone at a fixed concentration of 200 nM. Trans-4-{2-[4-(2,3-dichlorophenyl)-piperazin-1-yl]-ethyl}-N,N-dimethylcarbamoyl-cyclohexylamine hydrochloride will be studied at 3, 10, 30 and 100 nM in the absence or the presence of a fixed concentration of memantine (200 mM).

A total of 10 separate experiments will be required to establish a baseline for the effect of ouabain. Each concentration of memantine hydrochloride alone and trans-4-{2-[4-(2,3-dichlorophenyl)-piperazin-1-yl]-ethyl}-N,N-dimethylcarbamoyl-cyclohexylamine hydrochloride in the absence or the presence of a fixed concentration of memantine hydrochloride will be examined in 5 separate experiments.

Data Analysis. Statistical comparisons between the different treatment groups will be made by ANOVA with post hoc Fisher PLSD. A P value of <0.05 will be considered statistically significant. The primary outcome measure will be the effect of trans-4-{2-[4-(2,3-dichlorophenyl)-piperazin-1-yl]-ethyl}-N,N-dimethylcarbamoyl-cyclohexylamine hydrochloride, memantine hydrochloride or the combination of the two on the fraction of slices exhibiting cycling, or on the onset of cycling (e.g., whether trans-4-{2-[4-(2,3-dichlorophenyl)-piperazin-1-yl]-ethyl}-N,N-dimethylcarbamoyl-cyclohexylamine hydrochloride, memantine or the combination delay cycling).

It is anticipated that the described treatment regimes with trans-4-{2-[4-(2,3-dichlorophenyl)-piperazin-1-yl]-ethyl}-N,N-dimethylcarbamoyl-cyclohexylamine hydrochloride and memantine hydrochloride will show significant and surprising effectiveness in the treatment of, for example, bipolar depression and treatment resistant MDD, when compared to patients treated with each compound alone or with control.

### Example 4

### Cariprazine Hydrochloride and Memantine Hydrochloride for Anti-Manic Effects in the Mouse Amphetamine-Chlordiazepoxide Hyperactivity Model

Cariprazine hydrochloride (trans-4-{2-[4-(2,3-dichlorophenyl)-piperazin-1-yl]-ethyl}-N,N-dimethylcarbamoyl-cyclohexylamine hydrochloride) and memantine hydrochloride may be evaluated for anti-manic effects using the amphetamine/chlordiazepoxide (AMPH/CDP) hyperactivity test.

### Animals

Male C57B1/6J mice from Jackson Laboratories (Bar Harbor, Me.) may be used in this study. Mice may be received at 6-weeks of age. Upon receipt, mice may be assigned unique identification numbers (tail marked) and may be group housed with 4 mice/cage. All animals remained housed in groups of four during the remainder of the study. All mice may be acclimated to the colony room for at least two weeks prior to testing and may be subsequently tested at an average age of 8 weeks of age. During the period of acclimation, mice may be examined on a regular basis, handled, and weighed to assure adequate health and suitability. Mice may be maintained on a 12/12 light/dark cycle. The room temperature was maintained between 20 and 23° C. with a relative humidity maintained between 30% and 70%. Chow and water may be provided ad libitum for the duration of the study. In each test, animals may be randomly assigned across treatment groups.

### Drug Administration

The following compounds may be used:

d-Amphetamine sulfate (Sigma, 4.0 mg/kg) and chlordiazepoxide (CDP; Sigma, 2.5 mg/kg) may be dissolved in sterile water and may be administered intraperitoneally at a dose volume of 10 ml/kg.

Valproate (VPA; Sigma, Lot 064K1585, 400 mg/kg) was dissolved in sterile water and was administered intraperitoneally at a dose volume of 10 ml/kg 30 min prior to water or d-amphetamine/CDP mixture.

Cariprazine hydrochloride (0.03, 0.10, and 0.30 mg/kg) and memantine hydrochloride (0.5, 2.0, and 5.0 mg/kg) may be dissolved in sterile water and administered orally at a dose volume of 10 ml/kg 60 min prior to water or d-amphetamine/CDP mixture. The doses of cariprazine hydrochloride and memantine hydrochloride are expressed in mg free base per kg body weight.

### Methods

The open field test (OF) is used to assess both anxiety-like behavior and motor activity. The open field chambers are plexiglas square chambers (27.3×27.3×20.3 cm; Med Associates Inc., St Albans, Vt.) surrounded by infrared photobeam sources (16×16×16). Distance traveled is measured by consecutive beam breaks. Total distance traveled during the test session was used as an index of activity. After 60 minute pretreatment with water, cariprazine hydrochloride or memantine hydrochloride, or 30 minute pretreatment with valproate, mice may be injected with water or d-amphetamine/CDP mixture (‘mixture’) and placed in the OF chambers for a 60 min test session. At the end of each open field test session the OF chambers may be thoroughly cleaned.

### Statistical Analysis

Data was analyzed by analysis of variance (ANOVA) followed by Fisher PLSD post-hoc analysis when appropriate. An effect was considered significant if p<0.05. Outliers that fell above and below two standard deviations from the mean may be removed from the final analysis.

### Total Distance Traveled

The summary of the effects (e.g., the total distance traveled summed over the 60 minute test period) produced by different treatment regimens in the amphetamine-chlordiazepoxide mouse model of mania may establish that the effects from the combination of trans-4-{2-[4-(2,3-dichlorophenyl)-piperazin-1-yl]-ethyl}-N,N-dimethylcarbamoyl-cyclohexylamine hydrochloride and memantine hydrochloride are surprising and unexpected.

### Example 5

A clinical study will be conducted as a multicenter, randomized, double-blind, placebo-controlled, parallel-group, flexible-dose study. Patients will be selected who meet criteria that include those who (i) meet DSM-IV-TR criteria for bipolar I disorder (confirmed by the administration of the Structured Clinical Interview (SCID)), acute manic or mixed episode type with or without psychotic symptoms. and (ii) have a YMRS total score≧20 at Visit 1 and Visit 2 and a score of at least 4 on two of the following YMRS items: Irritability, Speech, Content, and Disruptive/Aggressive Behavior. Comorbid diagnoses such as conduct disorder, obsessive-compulsive disorder, anxiety disorders, and substance abuse will be allowed.

All patients meeting the eligibility criteria will be randomized (1:1 ratio) into one of two treatment groups:

(I) placebo,

(II) trans-4-{2-[4-(2,3-dichlorophenyl)-piperazin-1-yl]-ethyl}-N,N-dimethylcarbamoyl-cyclohexylamine hydrochloride,

(III) memantine hydrochloride, and

(IV) trans-4-{2-[4-(2,3-dichlorophenyl)-piperazin-1-yl]-ethyl}-N,N-dimethylcarbamoyl-cyclohexylamine hydrochloride and memantine hydrochloride.

### Efficacy Measurements

### Primary Efficacy Assessment

### Young Mania Rating Scale (YMRS)

The YMRS (see, e.g., Young et al., Br. J. Psychiatry, 133, 429-35, 1978) is an 11-item scale that assesses manic symptoms based on the patient's perception of his or her condition over the previous 48 hours, as well as the physician's clinical observations during the interview. The 11 items are elevated mood, increased motor activity-energy, sexual interest, sleep, irritability, rate and amount of speech, language-thought disorder, content, disruptive-aggressive behavior, appearance, and insight. The severity of the abnormality is rated on a five-point (0-4) or nine-point (0-8) scale; scoring between listed points is encouraged. Possible scores range from 0 to 60. This scale will be administered by a trained rater with expertise in evaluating manic patients. Assessments and ratings will be made by the same rater at approximately the same time of day.

### Secondary Efficacy Assessment

### Clinical Global Impressions-Severity (CGI-S)

The CGI-S (see, e.g., Guy ECDEU Assessment Manual for Psychopharmacology. Rockville, Md.: US Department of Health, Education, and Welfare, 218-22, 1976. Publication ADM 76-338) is a seven-point scale that measures the overall severity of the illness in comparison to the severity of other patients the physician has observed. This assessment will be made by a psychiatrist.

### Additional Efficacy Assessments

### Clinical Global Impressions-Improvement (CGI-I)

The Clinical Global Impressions-Improvement (CGI-I) (see, e.g., Guy ECDEU Assessment Manual for Psychopharmacology. Rockville, Md.: US Department of Health, Education, and Welfare, 218-22, 1976. Publication ADM 76-338) is a seven-point scale that measures the change from Baseline (Visit 2) in the overall severity of illness for the individual patient. The CGI-I will be assessed by a psychiatrist.

### Montgomery-Asberg Depression Rating Scale (MADRS)

The MADRS (see e.g., Montgomery and Asberg, Br. J. Psychiatry, 134, 382-9, 1979) is a clinician-rated scale that evaluates the patient's depressive symptomatology during the past week. Patients are to be rated on 10 items assessing feelings of sadness, lassitude, pessimism, inner tension, suicidality, reduced sleep or appetite, difficulty in concentration, and lack of interest. Each item will be scored on a seven-point scale with a score of 0 reflecting no symptoms and a score of 6 reflecting symptoms of maximum severity. This scale will be administered by a trained rater with adequate experience in the assessment of the patient's depressive symptomology.

### Positive and Negative Syndrome Scale (PANSS)

The PANSS (see, e.g., Kay et al. Schizophr. Bull., 13, 261-76, 1987) is a 30-item rating scale that was specifically developed to assess both the positive and negative symptom syndromes of patients with schizophrenia. The PANSS Total Score is rated based on a structured clinical interview with the patient and supporting clinical information obtained from family, hospital staff, or other reliable informants. Each item is scored on a seven-point (1-7) continuum and provides scores in nine clinical domains, including a positive syndrome, a negative syndrome, depression, a composite index, and general psychopathology. This scale will be administered by a trained, experienced psychiatric rater with expertise in the assessment of patients with bipolar disorder and schizophrenia.

It is anticipated that a treatment regime with trans-4-{2-[4-(2,3-dichlorophenyl)-piperazin-1-yl]-ethyl}-N,N-dimethylcarbamoyl-cyclohexylamine hydrochloride and memantine hydrochloride will show significant surprising effectiveness in the treatment of acute mania, e.g., acute mania associated with bipolar I disorder, when compared to patients treated with each compound alone or with control.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

The entire disclosures of all applications, patents and publications, cited herein, are hereby incorporated by reference in their entirety.

We claim:

1. A method of treating a condition selected from mania and bipolar disorder comprising administering to a patient in need thereof a therapeutically effective amount of an NMDA receptor antagonist, or a pharmaceutically acceptable salt thereof.

2. The method of claim 1, wherein the disorder is mania.

3. The method of claim 1, wherein the disorder is bipolar disorder.

4. The method of claim 2, wherein the mania is acute mania.

5. The method of claim 4, wherein the acute mania is associated with bipolar disorder.

6. The method of claim 5, wherein the acute mania is associated with bipolar I disorder.

7. The method of claim 6, wherein the acute mania is associated with bipolar II disorder.

8. The method of claim 1, wherein the NMDA receptor antagonist is selected from memantine, neramexane and pharmaceutically acceptable salts thereof.

9. The method of claim 8, wherein the NMDA receptor antagonist is memantine, or a pharmaceutically acceptable salt thereof.

10. The method of claim 9, wherein the NMDA receptor antagonist is memantine hydrochloride.

11. The method of claim 8, wherein the NMDA receptor antagonist is neramexane, or a pharmaceutically acceptable salt thereof.

12. The method of claim 9, wherein the NMDA receptor antagonist is neramexane hydrochloride.

13. The method of claim 9, wherein the NMDA receptor antagonist is neramexane mesylate.

14. A method of treating a condition selected from mania, bipolar disorder and schizophrenia, comprising administering to a patient in need thereof a therapeutically effective amount of an NMDA receptor antagonist, or a pharmaceutically acceptable salt thereof, and a compound of formula (I), or a pharmaceutically acceptable salt thereof: 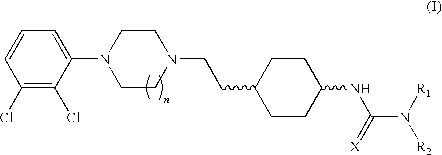
wherein
R₁ and R₂ are each, independently hydrogen, alkyl, alkenyl, aryl, cycloalkyl, or aroyl, or R₁ and R₂ form a heterocyclic ring with the adjacent nitrogen atom;
X is O or S; and
n is 1 or 2.

15. The method of claim 14, wherein the compound of formula (I) is trans-1-{4-[2-[4-(2,3-dichlorophenyl)-piperazin-1-yl]-ethyl]-cyclohexyl}-3,3-dimethyl-urea, or a pharmaceutically acceptable salt thereof.

16. The method of claim 15, wherein the compound of formula (I) is trans-1-{4-[2-[4-(2,3-dichlorophenyl)-piperazin-1-yl]-ethyl]-cyclohexyl}-3,3-dimethyl-urea hydrochloride.

17. The method of claim 15, wherein the NMDA receptor antagonist is selected from the group consisting of memantine, neramexane and pharmaceutically acceptable salts and solvates thereof.

18. The method of claim 15, wherein the NMDA receptor antagonist is memantine or a pharmaceutically acceptable salt thereof.

19. The method of claim 15, wherein the NMDA receptor antagonist is memantine hydrochloride.

20. The method of claim 15, wherein the NMDA receptor antagonist is neramexane, or a pharmaceutically acceptable salt thereof.

21. The method of claim 15, wherein the NMDA receptor antagonist is neramexane hydrochloride.

22. The method of claim 15, wherein the NMDA receptor antagonist is neramexane mesylate.

23. The method of claim 14, wherein the disorder is mania.

24. The method of claim 14, wherein the disorder is bipolar disorder.

25. The method of claim 23, wherein the mania is acute mania.

26. The method of claim 25, wherein the acute mania is associated with bipolar disorder.

27. The method of claim 26, wherein the acute mania is associated with bipolar I disorder.

28. The method of claim 26, wherein the acute mania is associated with bipolar II disorder.

29. The method of claim 14, wherein the disorder is schizophrenia.

30. The method of claim 29, wherein the negative symptoms of schizophrenia are treated.

31. The method of claim 29, wherein the positive symptoms of schizophrenia are treated

32. The method of claim 29, wherein the cognitive symptoms of schizophrenia are treated.

33. The method of claim 29, wherein the affective and residual symptoms of schizophrenia are treated.

34. The method of claim 29, wherein the secondary social and occupational dysfunctions of schizophrenia are treated.

35. A pharmaceutical composition comprising
(i) an NMDA receptor antagonist,
(ii) a compound of formula (I), or a pharmaceutically acceptable salt thereof: 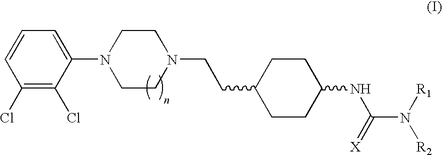
wherein
R₁ and R₂ are each, independently hydrogen, alkyl, alkenyl, aryl, cycloalkyl, or aroyl, or R₁ and R₂ form a heterocyclic ring with the adjacent nitrogen atom;
X is O or S; and
n is 1 or 2;
and pharmaceutically acceptable salts and solvates thereof, and
(iii) a pharmaceutically acceptable carrier.

36. The composition of claim 35, wherein the compound of formula (I) is trans-1-{4-[2-[4-(2,3-dichlorophenyl)-piperazin-1-yl]-ethyl]-cyclohexyl}-3,3-dimethyl-urea, or a pharmaceutically acceptable salt thereof.

37. The composition of claim 36, wherein the compound of formula (I) is trans-1-{4-[2-[4-(2,3-dichlorophenyl)-piperazin-1-yl]-ethyl]-cyclohexyl}-3,3-dimethyl-urea hydrochloride.

38. The composition of any one of claims 36, wherein the NMDA receptor antagonist is selected from memantine, neramexane and pharmaceutically acceptable salts thereof.

39. The composition of claim 38, wherein the NMDA receptor antagonist is memantine or a pharmaceutically acceptable salt thereof.

40. The composition of claim 39, wherein the NMDA receptor antagonist is memantine hydrochloride.

41. The composition of claim 38, wherein the NMDA receptor antagonist is neramexane, or a pharmaceutically acceptable salt thereof.

42. The composition of claim 41, wherein the NMDA receptor antagonist is neramexane hydrochloride.

43. The composition of claim 41, wherein the NMDA receptor antagonist is neramexane mesylate.
